(19) United States
(12) Patent Application Publication
(10) Pub. No.: US 2023/0320615 A1
(43) Pub. Date: Oct. 12, 2023

(54) ARRAY COIL AND MANUFACTURING METHOD

(71) Applicant: CANON MEDICAL SYSTEMS CORPORATION, Otawara-shi (JP)

(72) Inventors: Masahiro FUKUSHIMA, Utsunomiya (JP); Kazuya OKAMOTO, Saitama (JP)

(73) Assignee: CANON MEDICAL SYSTEMS CORPORATION, Otawara-shi (JP)

(21) Appl. No.: 18/193,138

(22) Filed: Mar. 30, 2023

(30) Foreign Application Priority Data
Apr. 8, 2022 (JP) 2022-064644

(51) Int. Cl. A61B 5/055

(52) U.S. Cl. CPC A61B 5/055; A61B 2017/00911

(57) ABSTRACT

In a medical image diagnostic apparatus according to an embodiment, an array coil includes a first substrate and a second substrate. At least one coil element is formed on the first substrate. The second substrate is a substrate different from the first substrate, and is laminated on the first substrate. At least one coil element is formed on the second substrate. A first coil element formed on the first substrate intersects with a second coil element formed on the second substrate in plan view from a laminating direction from the second substrate laminated on the first substrate toward the first substrate.

### CROSS-REFERENCE TO RELATED APPLICATIONS

This application is based upon and claims the benefit of priority from Japanese Patent Application No. 2022-064644, filed on Apr. 8, 2022, the entire contents of which are incorporated herein by reference.

### FIELD

Embodiments described herein relate generally to an array coil and a manufacturing method.

### BACKGROUND

In the related art, there is known a magnetic resonance imaging (MRI) apparatus configured to excite a nuclear spin of a biological tissue placed in a strong static magnetic field with a high frequency signal having the Larmor frequency thereof, and reconstruct image data based on a magnetic resonance signal (MR signal) generated from a subject following the excitation. The MRI apparatus emits, to the subject placed in the static magnetic field, a high-frequency magnetic field generated by an RF coil to which an RF signal amplified by a Radio Frequency (RF) amplifier is supplied.

For example, there is known a technique of manufacturing an array coil (RF coil) including a plurality of coil elements by forming a coil pattern on a substrate. However, in a case of forming a coil pattern in which coil elements intersect with each other on the same plane, a solid wire to be a jumper is required to be manually soldered after forming the coil pattern on the substrate so that adjacent coil elements are prevented from being brought into electrical contact with each other. Furthermore, there has been the problem that, when adjacent coil elements are present, work of adjusting the array coil such as geometry decoupling is complicated, for example.

### BRIEF DESCRIPTION OF THE DRAWINGS

FIG. 1 is a diagram illustrating an example of a configuration of a Magnetic Resonance Imaging (MRI) apparatus according to an embodiment;

FIG. 2 is a diagram illustrating an example of a configuration of a unit flexible substrate on which coil elements are formed according to the embodiment;

FIG. 3 is a diagram illustrating an example of a configuration of an array coil that is formed by laminating unit flexible substrates according to the embodiment;

FIG. 4 is a diagram illustrating another example of a configuration of the array coil that is formed by laminating the unit flexible substrates according to the embodiment;

FIG. 5 is a diagram for explaining geometry decoupling for the array coil according to the embodiment;

FIG. 6 is a flowchart illustrating an example of a procedure of a manufacturing step of the array coil according to the embodiment; and

FIG. 7 is a diagram illustrating an example of a configuration of an array coil that is formed by manually performing soldering on a flexible substrate unlike the array coil according to the embodiment.

### DETAILED DESCRIPTION

An array coil according to an embodiment includes a first substrate and a second substrate. At least one coil element is formed on the first substrate. The second substrate is a substrate different from the first substrate, and is laminated on the first substrate. At least one coil element is formed on the second substrate. A first coil element formed on the first substrate intersects with a second coil element formed on the second substrate in plan view from a laminating direction from the second substrate laminated on the first substrate toward the first substrate.

The following describes the array coil and a manufacturing method according to respective embodiments with reference to the drawings. In the following description, a constituent element having the same or substantially the same function as a function that has been already described with reference to the drawing is denoted by the same reference numeral, and redundant description will be repeated only when it is necessary. Even in a case in which the same portion is represented, dimensions or ratios thereof may be different between the drawings in some cases.

### Embodiment

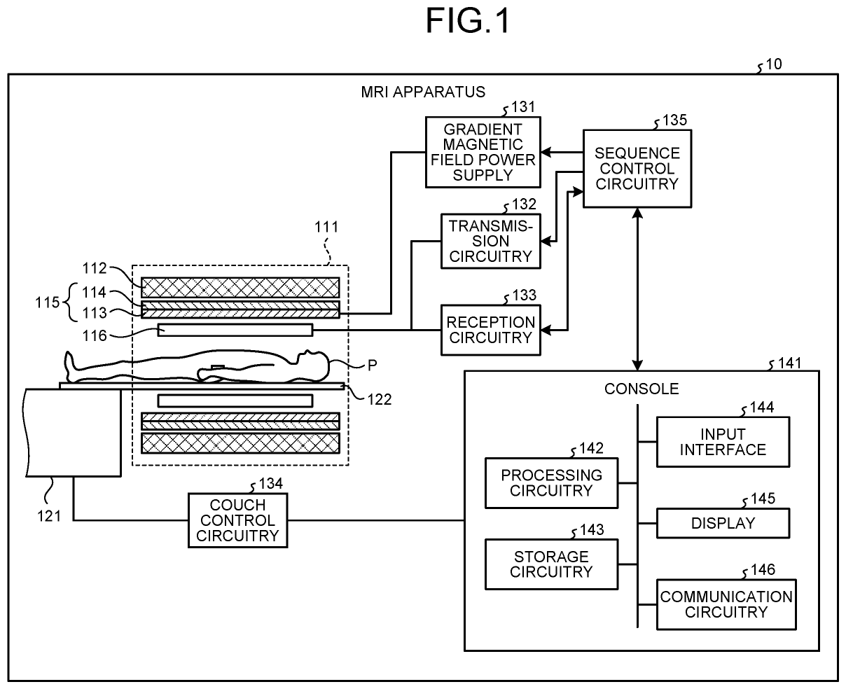

FIG. 1 is a diagram illustrating an example of a configuration of a Magnetic Resonance Imaging (MRI) apparatus 10 according to the embodiment. The MRI apparatus 10 is an apparatus configured to reconstruct an image based on a magnetic resonance signal acquired by imaging, which is performed by emitting a high-frequency magnetic field to a subject P placed in a static magnetic field. As illustrated in FIG. 1, the MRI apparatus 10 includes a magnet stand 111 and a couch 121. The magnet stand 111 includes a static magnetic field magnet 112, a gradient coil unit 115, and an RF coil 116. In FIG. 1, an internal configuration of the magnet stand 111 is exemplified in a vertical cross-sectional view. The MRI apparatus 10 does not include the subject P (for example, a human body). The configuration illustrated in FIG. 1 is merely an example. For example, part of or the entire sequence control circuitry 135 and console 141 may be configured to be appropriately integrated with each other, or may be configured to be appropriately separated from each other. For example, the MRI apparatus 10 is installed in an MR imaging room.

The gradient coil unit 115 includes a main coil 113 and a shield coil 114. The MRI apparatus 10 includes a gradient magnetic field power supply 131, transmission circuitry 132, reception circuitry 133, couch control circuitry 134, the sequence control circuitry 135, and the console 141.

The static magnetic field magnet 112 has a substantially cylindrical shape, and generates a static magnetic field in a bore (a space inside a cylinder of the static magnetic field magnet 112) including an imaging region of the subject P. The static magnetic field magnet 112 may be a superconducting magnet or a permanent magnet.

The gradient coil unit 115 has a substantially cylindrical shape, and is held by a support structure such as vibration-proof rubber on an inner side of the static magnetic field magnet 112. The gradient coil unit 115 includes the main coil 113 that applies (generates) a gradient magnetic field in directions orthogonal to each other by a current supplied from the gradient magnetic field power supply 131, and the shield coil 114 that cancels a leakage magnetic field of the main coil 113.

The couch 121 includes a couchtop 122 on which the subject P is placed, and inserts the couchtop 122 into a cavity (imaging port) of the gradient coil unit 115 in a state in which the subject P is placed thereon under control by the couch control circuitry 134. The couch control circuitry 134 drives the couch 121 to move the couchtop 122 in a longitudinal direction and an upper and lower direction under control by the console 141.

The Radio Frequency (RF) coil 116 is arranged on an inner side of the gradient coil unit 115, and receives supply of an RF pulse from the transmission circuitry 132 to generate a high-frequency magnetic field. Furthermore, the RF coil 116 receives a magnetic resonance signal emitted from the subject P due to influence of the high-frequency magnetic field, and outputs the received magnetic resonance signal to the reception circuitry 133. The RF coil 116 may be constituted of a transmission coil and a reception coil, which are separated from each other.

The transmission circuitry 132 supplies, to the RF coil 116, a high frequency pulse modulated into the Larmor frequency (also referred to as a magnetic resonance frequency) under control by the sequence control circuitry 135. In the present embodiment, the high frequency pulse modulated into the Larmor frequency (also referred to as a magnetic resonance frequency) may be referred to as an RF pulse or an RF signal in some cases. The magnetic resonance frequency is set in advance in accordance with a gyromagnetic ratio corresponding to an atom as a magnetic resonance target and magnetic flux density of a static magnetic field. That is, the frequency of the RF signal varies depending on a nuclide as a measurement target in measurement based on the RF signal. In a case in which the magnetic flux density of the static magnetic field is 1.5 T, the magnetic resonance frequency is about 64 MHz. In a case in which the magnetic flux density of the static magnetic field is 3 T, the magnetic resonance frequency is about 128 MHz. For example, the transmission circuitry 132 includes an oscillation unit, a phase selection unit, a frequency conversion unit, an amplitude modulation unit, an RF amplifier, and the like.

The oscillation unit generates an RF pulse of a resonance frequency specific to a target atomic nucleus in the static magnetic field. The oscillation unit corresponds to a quartz oscillator including oscillator circuitry using a crystal transducer element, a frequency multiplier, and the like. That is, the quartz oscillator is an oscillator configured by using, as source oscillation, oscillation (system clock) obtained by multiplying an oscillation frequency of the crystal transducer element by an integer using a frequency multiplier. The oscillator circuitry does not necessarily use the crystal transducer element but may use another transducer element. The oscillation unit may be disposed in processing circuitry 142, or may be mounted on the console 141. At this point, the oscillation unit becomes source oscillation related to the entire control of the MRI apparatus 10.

The phase selection unit selects a phase of the RF pulse generated by the oscillation unit.

The frequency conversion unit converts the frequency of the RF pulse output from the phase selection unit.

The amplitude modulation unit modulates amplitude of the RF pulse output from the frequency conversion unit in accordance with a sinc function, for example.

The RF amplifier amplifies the RF pulse having the magnetic resonance frequency output from the amplitude modulation unit, and supplies the RF pulse to the RF coil 116 via a duplexer (not illustrated). For example, the RF amplifier amplifies the RF pulse to ten or more kilowatts to several tens of kilowatts.

The reception circuitry 133 detects the magnetic resonance signal output from the RF coil 116, and generates magnetic resonance data based on the detected magnetic resonance signal. Specifically, the reception circuitry 133 generates the magnetic resonance data by digitally converting the magnetic resonance signal received by the RF coil 116. The reception circuitry 133 also transmits the generated magnetic resonance data to the sequence control circuitry 135.

The sequence control circuitry 135 executes a pulse sequence to image the subject P by driving the gradient magnetic field power supply 131, the transmission circuitry 132, and the reception circuitry 133 based on sequence information transmitted from the console 141. Herein, the sequence information is information defining a procedure to perform imaging. The sequence information defines, as the pulse sequence, strength of a current supplied from the gradient magnetic field power supply 131 to the main coil 113 and a timing for supplying the current, strength of the RF pulse supplied from the transmission circuitry 132 to the RF coil 116 and a timing for applying the RF pulse, a timing at which the reception circuitry 133 detects the magnetic resonance signal, and the like. For example, the sequence control circuitry 135 is implemented by a processor.

The sequence information may include a nuclide as a measurement target or a frequency of the RF pulse (input signal) supplied to the RF coil 116.

Furthermore, when the sequence control circuitry 135 receives the magnetic resonance data from the reception circuitry 133 as a result of imaging the subject P by driving the gradient magnetic field power supply 131, the transmission circuitry 132, and the reception circuitry 133, the sequence control circuitry 135 transfers the received magnetic resonance data to the console 141.

Each of the transmission circuitry 132, the reception circuitry 133, the couch control circuitry 134, and the like is similarly constituted of electronic circuitry such as a processor as described above.

The console 141 is a computer that controls the MRI apparatus 10. The console 141 performs overall control for the MRI apparatus 10, and generates an image, for example. The console 141 includes the processing circuitry 142, storage circuitry 143, an input interface 144, a display 145, and communication circuitry 146.

The processing circuitry 142 includes a processor such as a CPU, and a memory such as a ROM and a RAM as hardware resources. The processing circuitry 142 executes respective functions of the MRI apparatus 10 by a processor that executes a computer program loaded into the memory. The processing circuitry 142 performs overall control for the MRI apparatus 10, and controls imaging, generation of an image, display of an image, and the like. For example, the processing circuitry 142 receives an input of an imaging condition (an imaging parameter and the like) on a GUI, and generates sequence information in accordance with the received imaging condition. The processing circuitry 142 also transmits the generated sequence information to the sequence control circuitry 135. The processing circuitry 142 also receives the magnetic resonance data from the sequence control circuitry 135, and stores the received magnetic resonance data in the storage circuitry 143. The processing circuitry 142 also reads out k-space data from the storage circuitry 143, and generates an image by performing reconstruction processing such as Fourier transformation on the k-space data that has been read out. That is, the processing circuitry 142 reconstructs the image based on the magnetic resonance signal acquired by imaging, which is performed by emitting a high-frequency magnetic field to the subject P placed in the static magnetic field.

The storage circuitry 143 stores various pieces of information used by the processing circuitry 142. Specifically, the storage circuitry 143 stores the magnetic resonance data received by the processing circuitry 142, the k-space data disposed in a k-space by the processing circuitry 142, image data generated by the processing circuitry 142, and the like. The storage circuitry 143 also stores various computer programs executed by the processing circuitry 142, and various pieces of setting information. Specifically, the storage circuitry 143 stores a computer program that supports positioning of an imaging range, a computer program related to signal processing of the magnetic resonance data, and the like. For example, the storage circuitry 143 is implemented by a semiconductor memory element such as a RAM, ROM, and a flash memory, a hard disk, an optical disc, and the like.

The input interface 144 receives various input operations from an operator, and converts the received input operation into an electric signal to be output to the processing circuitry 142. For example, the input interface 144 is a selection device such as a pointing device including a mouse, a trackball, and the like, or an input device such as a keyboard. Examples of the input interface 144 also include processing circuitry for an electric signal that receives an electric signal corresponding to the input operation from external input equipment that is disposed separately from the console 141, and outputs the electric signal to the processing circuitry 142.

Under control by the processing circuitry 142, the display 145 displays a Graphical User Interface (GUI) for receiving an input related to adjustment or setting of the imaging condition, an image generated by the processing circuitry 142, and the like. As the display 145, various optional displays can be appropriately used. For example, as the display 145, a Liquid Crystal Display (LCD), a Cathode Ray Tube (CRT) display, an Organic Electro Luminescence Display (OELD), or a plasma display can be used.

The display 145 may be disposed at any place. For example, the display 145 may be disposed in an imaging room, an operation room, or the like. The display 145 may also be disposed on the magnet stand 111. The display 145 may be a desktop type, or may be constituted of a tablet terminal and the like that can communicate with a main body of the console 141 in a wireless manner. As the display 145, one or two or more projectors may be used.

The communication circuitry 146 communicates with an external apparatus such as an information processing apparatus 30 via a network. The communication circuitry 146 is, for example, a communication interface such as a network card, a network adapter, and a Network Interface Controller (NIC).

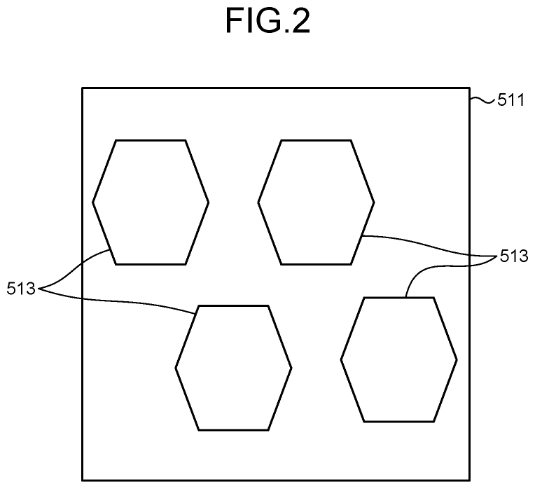

FIG. 2 is a diagram illustrating an example of a configuration of a unit flexible substrate 511 on which coil elements 513 are formed according to the embodiment.

As illustrated in FIG. 2, a plurality of the coil elements 513 are formed on the unit flexible substrate 511. On the unit flexible substrate 511, the coil element 513 are disposed to be separated from each other. In other words, on the unit flexible substrate 511, each of the coil elements 513 does not intersect with the adjacent coil element 513. Herein, “adjacent coil elements 513 are separated from each other” or “each of the coil elements 513 does not intersect with the adjacent coil element 513” means that a pair of adjacent coil elements 513 are not electrically connected with each other, that is, they are not short-circuited.

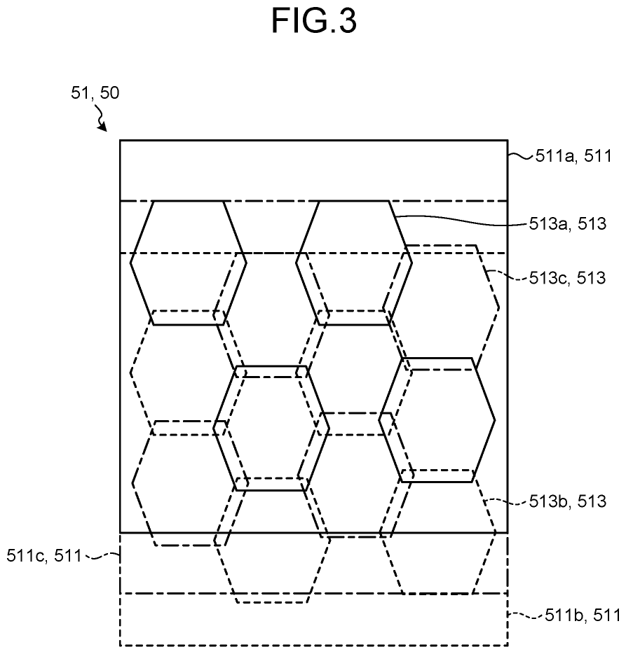

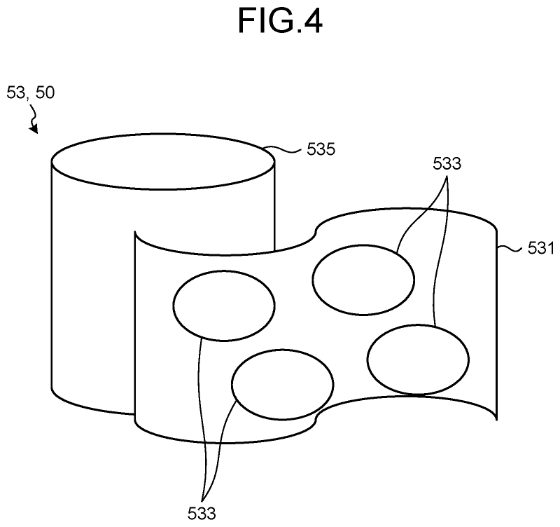

In the example illustrated in FIG. 3, each of the coil elements 513 has a hexagonal shape. Each of the coil elements 513 may have another shape such as an elliptic shape similarly to a coil element 533 in FIG. 4.

By way of example, the unit flexible substrate 511 is formed of a material having flexibility such as polyimide or polycarbonate. The unit flexible substrate 511 may be formed of another material.

FIG. 3 is a diagram illustrating an example of a configuration of an array coil 50 that is formed by laminating unit flexible substrates 511 according to the embodiment. The array coil 50 is an example of the RF coil 116 described above. FIG. 3 exemplifies a planar array coil 51 as an example of the array coil 50.

The planar array coil 51 includes the unit flexible substrates 511. FIG. 3 exemplifies a first unit flexible substrate 511a, a second unit flexible substrate 511b, and a third unit flexible substrate 511c as the unit flexible substrates 511 included in the planar array coil 51. FIG. 3 exemplifies a case in which the second unit flexible substrate 511b is laminated on the third unit flexible substrate 511c, and the first unit flexible substrate 511a is laminated on the second unit flexible substrate 511b. A plurality of coil elements 513a are formed on the first unit flexible substrate 511a. A plurality of coil elements 513b are formed on the second unit flexible substrate 511b. A plurality of coil elements 513c are formed on the third unit flexible substrate 511c.

Herein, an optional unit flexible substrate 511 (for example, the first unit flexible substrate 511a) of the unit flexible substrates 511 is an example of a first substrate. A coil element (for example, each of the coil elements 513a) formed on the first substrate of the unit flexible substrates 511 is an example of a first coil element. A substrate other than the first substrate (for example, the second unit flexible substrate 511b or the third unit flexible substrate 511c) of the unit flexible substrates 511 is an example of a second substrate. A coil element (for example, each of the coil elements 513b or the coil elements 513c) formed on the second substrate of the unit flexible substrates 511 is an example of a second coil element.

The number of the unit flexible substrates 511 of the planar array coil 51 may be appropriately determined based on a size of each of the unit flexible substrates 511 and a size of the planar array coil 51.

Specifically, the planar array coil 51 is formed by laminating the unit flexible substrates 511. In other words, the planar array coil 51 is divided into the unit flexible substrates 511 in a thickness direction. In the planar array coil 51, the unit flexible substrates 511 may be fixed to each other with an adhesive agent, for example.

As described above, on the unit flexible substrate 511, each of the coil elements 513 does not intersect with the adjacent coil element 513. On the other hand, as illustrated in FIG. 3, the coil elements 513 of the planar array coil 51 intersect with each other in plan view from a direction perpendicular to a principal plane of the planar array coil 51, that is, viewed from a laminating direction of the unit flexible substrate 511. In other words, the planar array coil 51 is formed by laminating the unit flexible substrates 511 so that the coil elements 513 respectively formed on different unit flexible substrates 511 intersect with each other in plan view.

In the example illustrated in FIG. 3, the coil element 513a of the first unit flexible substrate 511a intersects with the coil element 513b of the second unit flexible substrate 511b in plan view. Similarly, the coil element 513a of the first unit flexible substrate 511a intersects with the coil element 513b of the second unit flexible substrate 511b in plan view. Similarly, the coil element 513b of the second unit flexible substrate 511b intersects with the coil element 513c of the third unit flexible substrate 511c in plan view.

In the planar array coil 51, each of the unit flexible substrates 511 may be formed of a material not having flexibility. That is, each of the unit flexible substrates 511 of the planar array coil 51 is not necessarily a flexible substrate.

The array coil 50 formed of the unit flexible substrates 511 is not limited to the planar array coil 51. FIG. 4 is a diagram illustrating another example of the configuration of the array coil 50 that is formed by laminating the unit flexible substrates 511 according to the embodiment. FIG. 4 exemplifies a volume array coil 53 as an example of the array coil 50.

The volume array coil 53 includes a plurality of unit flexible substrates 531. The unit flexible substrates 531 have the same configuration as that of the unit flexible substrates 511 in FIG. 3.

FIG. 4 exemplifies only one unit flexible substrate 531 as the unit flexible substrates 531 included in the volume array coil 53. The number of the unit flexible substrates 531 of the volume array coil 53 may be appropriately determined based on a size of each of the unit flexible substrates 531 and a size of the volume array coil 53. A plurality of the coil elements 533 are formed on the unit flexible substrate 531.

Each of the coil elements 533 has the same configuration as that of each of the coil elements 513 in FIG. 2 and FIG. 3. In the example illustrated in FIG. 4, each of the coil elements 533 has an elliptic shape. Each of the coil elements 533 may have another shape such as a hexagonal shape similarly to the coil element 513 in FIG. 3.

Herein, an optional unit flexible substrate 531 of the unit flexible substrates 531 is an example of the first substrate. Each of the coil elements 533 formed on the first substrate of the unit flexible substrates 531 is an example of the first coil element. A substrate other than the first substrate of the unit flexible substrates 531 is an example of the second substrate. Each of the coil elements 533 formed on the second substrate of the unit flexible substrates 531 is an example of the second coil element.

Specifically, the volume array coil 53 is formed by laminating and winding the unit flexible substrates 531 around a shaft 535. In other words, the volume array coil 53 is divided into the unit flexible substrates 531 in a thickness direction, that is, in a radial direction of the shaft 535. In the volume array coil 53, the unit flexible substrates 531 may be fixed to each other with an adhesive agent, for example.

As described above, on the unit flexible substrate 531, each of the coil elements 533 does not intersect with the adjacent coil element 533. On the other hand, the coil elements 533 of the volume array coil 53 intersect with each other in plan view from the radial direction of the shaft 535. In other words, the volume array coil 53 is formed by laminating the unit flexible substrates 531 so that the coil elements 533 respectively formed on different unit flexible substrates 531 intersect with each other in plan view.

FIG. 2 and FIG. 4 respectively exemplify unit flexible substrates 511 and 531 on which four coil elements 513 and 533 are formed, but the embodiment is not limited thereto. The number of the coil elements 513 and 533 formed on the unit flexible substrates 511 and 531 may be appropriately designed depending on a high-frequency magnetic field required for the array coil 50. The number of the coil elements 513 and 533 may be one, two, three, or five or more.

A shape and a size of each of the coil elements 513 and 533, and a pattern of the coil elements 513 and 533 such as an interval between the coil elements are appropriately designed depending on a high-frequency magnetic field required for the array coil 50 as described later.

On each of the unit flexible substrates 511 and 531, shapes of the respective coil elements 513 and 533 are uniform, for example, but may be different from each other. On each of the unit flexible substrates 511 and 531, sizes of the respective coil elements 513 and 533 are uniform, for example, but may be different from each other.

The size of each of the coil elements 513 and 533 may be appropriately designed depending on a position at which the array coil 50 is disposed. By way of example, the coil elements 513 and 533 of the array coil 50 targeted for a surface of a body of the subject P may be larger than the coil elements 513 and 533 of the array coil 50 targeted for a portion deeper than the surface of the body of the subject P.

On the unit flexible substrates 511 and 531, a hole part (not illustrated) is disposed at a position interfering with another constituent element of the array coil 50 such as tuning circuitry, matching circuitry, a capacitor, and another substrate of the array coil 50.

The array coil 50 may include a region in which the unit flexible substrates 511 and 531 do not overlap with each other. In other words, the unit flexible substrates 511 and 531 are not necessarily laminated over the entire array coil 50.

In the array coil 50, the unit flexible substrates 511 may be common, or at least one unit flexible substrate 511 of the unit flexible substrates 511 may be different from the other unit flexible substrates 511.

Herein, “the unit flexible substrates 511 and 531 are different” may mean that arrangement of the coil elements 513 and 533 on the respective unit flexible substrates 511 and 531 is different. The arrangement of the coil elements 513 and 533 is defined by at least one of the number and position of the coil elements 513 and 533.

By way of example, in a case in which the array coil 50 is applied to a body coil, the coil elements 513 and 533 are arranged on the respective unit flexible substrates 511 and 531 so that distribution of the coil elements 513 and 533 in the array coil 50 becomes uniform. By way of example, in a case in which the array coil 50 is applied to a head coil, the coil elements 513 and 533 are arranged on the respective unit flexible substrates 511 and 531 so that the coil elements 513 and 533 are densely arranged on a head side of the subject P of the array coil 50. In this way, by differently arranging the coil elements 513 and 533 on the respective unit flexible substrates 511 and 531, resolution of the array coil 50 can be distributed.

“The unit flexible substrates 511 and 531 are different” may mean that thicknesses of the respective unit flexible substrates 511 and 531 are different, for example.

Regarding the unit flexible substrates 511 and 531, the thicknesses of the substrates may be uniform, or there may be distribution in the thicknesses of the substrates depending on the arrangement of the coil elements 513 and 533, for example.

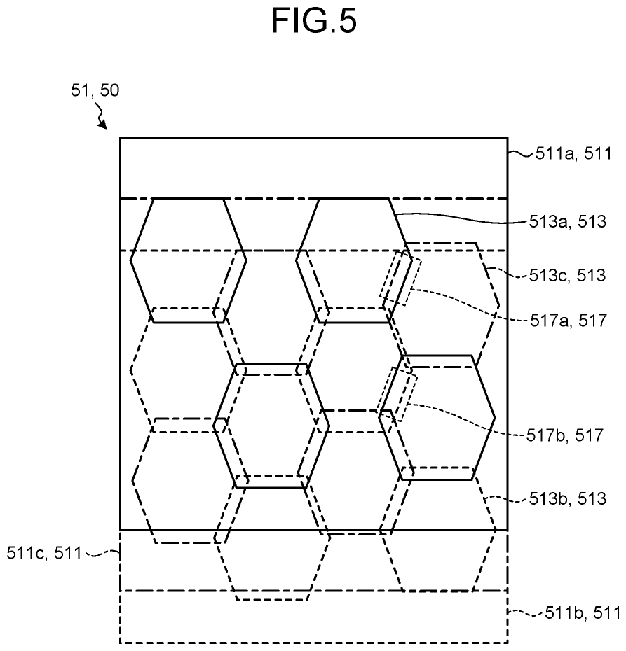

The following describes decoupling for the array coil 50 according to the embodiment. FIG. 5 is a diagram for explaining geometry decoupling for the array coil 50 according to the embodiment; The planar array coil 51 in FIG. 5 exemplifies a state in which spacers 517 are inserted into the planar array coil 51 in FIG. 3. Herein, the spacer 517 is an example of an adjustment member.

For simplification of explanation, the following exemplifies a case of performing geometry decoupling on the planar array coil 51 in FIG. 3, but the same is applied to the volume array coil 53 in FIG. 4.

As described above, the array coil 50 according to the embodiment can be formed by laminating the unit flexible substrates 511. Due to this, in the array coil 50 according to the embodiment, the coil element 513 can be moved for each of the unit flexible substrates 511. Thus, geometry decoupling for the array coil 50 according to the embodiment can be implemented by interposing the spacer 517 between the unit flexible substrates 511 in the laminating direction. The geometry decoupling for the array coil 50 according to the embodiment can also be implemented by adjusting a position with respect to the other unit flexible substrate 511, that is, a position to be bonded.

The spacer 517 is formed of a non-magnetic body that does not shield a magnetic field such as glass, glass epoxy resin, or Teflon (registered trademark), for example. The spacer 517 has a plate shape, for example.

Specifically, the spacer 517 is arranged at a position between the coil elements 513 as adjustment targets intersecting with each other in plan view between the unit flexible substrates 511 that are adjacent to each other in the laminating direction. Due to this, a distance (thickness) between the coil elements 513 respectively formed on the unit flexible substrates 511 that are different from each other is adjusted, so that an amount of magnetic flux passing through the coil element 513 as the adjustment target can be adjusted.

In the example illustrated in FIG. 5, a spacer 517a (spacer 517) is arranged at a position at which the coil element 513a of the first unit flexible substrate 511a intersects with the coil element 513c of the third unit flexible substrate 511c in plan view. Herein, the spacer 517a may be interposed between the first unit flexible substrate 511a and the second unit flexible substrate 511b, or may be interposed between the second unit flexible substrate 511b and the third unit flexible substrate 511c. That is, in a case of performing decoupling of the coil elements 513 between the unit flexible substrates 511 that are not adjacent to each other in the laminating direction, the spacer 517 may be inserted between an optional pair of the unit flexible substrates 511 between a pair of the unit flexible substrates 511 on which the coil elements 513 as targets are disposed.

In the example illustrated in FIG. 5, a spacer 517b (spacer 517) is arranged at a position at which the coil element 513a of the first unit flexible substrate 511a intersects with the coil element 513b of the second unit flexible substrate 511b in plan view. The spacer 517b is interposed between the first unit flexible substrate 511a and the second unit flexible substrate 511b, for example.

The spacer 517 does not necessarily have a plate shape, but may have another shape such as a linear shape. The spacer 517 may be inserted between the entire unit flexible substrates 511. That is, the spacer 517 may be used for changing a distance between the coil elements 513 that are adjacent to each other in the laminating direction, or may be used for changing a distance itself between the unit flexible substrates 511 that are adjacent to each other in the laminating direction.

The geometry decoupling for the planar array coil 51 according to the embodiment is not necessarily performed by using the spacer 517, but may be performed by using the unit flexible substrates 511 having different thicknesses. Specifically, the geometry decoupling may be implemented by replacing at least one of the unit flexible substrates 511 on which the coil elements 513 as the adjustment targets are formed with the unit flexible substrate 511 having a different thickness and having the same arrangement of the coil elements 513. “The thickness of the unit flexible substrate 511 is different” may mean that the thickness of the entire unit flexible substrate 511 is different, or the thickness at a position intersecting with the coil element 513 of the other unit flexible substrate 511 when being laminated is locally different. Herein, the unit flexible substrate 511 to be replaced can be represented as an example of the adjustment member.

The geometry decoupling can also be implemented by changing a wire width of the coil element 513 such as shaving part of the coil element 513. In this case, the geometry decoupling may be implemented by replacing at least one of the unit flexible substrates 511 on which the coil elements 513 as the adjustment targets are formed with the unit flexible substrate 511 in which the wire width of the coil element 513 is different, that is, inductance is different, and the arrangement of the coil elements 513 is the same.

In this way, the planar array coil 51 according to the embodiment is formed by laminating the unit flexible substrates 511, so that the geometry decoupling can be implemented by replacing part of the unit flexible substrates 511. Additionally, work of adjusting the wire width can be performed on the unit flexible substrate 511 that is not laminated, so that the adjustment work can be facilitated.

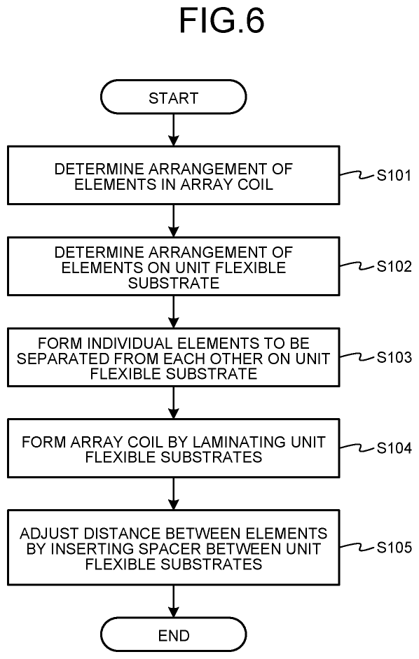

The following describes a manufacturing method for the array coil 50 (RF coil 116) according to the embodiment. FIG. 6 is a flowchart illustrating an example of a procedure of a manufacturing step of the array coil 50 according to the embodiment.

First, the arrangement of the coil elements 513 and 533 in the array coil 50 is determined (S101). The arrangement of the coil elements 513 and 533 may be appropriately designed depending on a high-frequency magnetic field required for the array coil 50, for example.

Next, the arrangement of the coil elements 513 and 533 on the respective unit flexible substrates 511 and 531 is determined (S102). Specifically, the arrangement of the coil elements 513 and 533 in the array coil 50 is divided, and the arrangement of the coil elements 513 and 533 on the respective unit flexible substrates 511 and 531 is determined. At this point, among the coil elements 513 and 533 in the array coil 50, the coil elements 513 and 533 that intersect with each other when being formed on the same plane are arranged on the unit flexible substrates 511 and 531 different from each other.

The individual coil elements 513 and 533 are formed to be separated from each other on the unit flexible substrates 511 and 531 (S103). Specifically, in accordance with the arrangement determined at S102, the coil elements 513 and 533 are printed on the respective unit flexible substrates 511 and 531.

Thereafter, the array coil 50 is formed by laminating the unit flexible substrates 511 and 531 on which the coil elements 513 and 533 are formed (S104). Additionally, for example, a spacer 517 is inserted between the unit flexible substrates 511 and 531 to perform decoupling for adjusting the distance between the coil elements 513 and 533 (S105).

In this way, the array coil 50 according to the embodiment includes the laminated unit flexible substrates 511 and 531. Additionally, at least one of the coil elements 513 and 533 is formed on each of the unit flexible substrates 511 and 531. Herein, the first coil element formed on the first substrate intersects with the second coil element formed on the second substrate in plan view from the laminating direction from the second substrate laminated on the first substrate toward the first substrate.

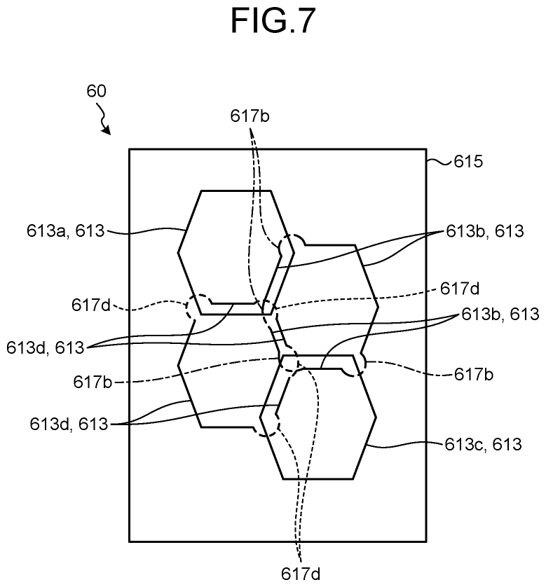

FIG. 7 is a diagram illustrating an example of a configuration of an array coil 60 that is formed by manually performing soldering on a flexible substrate 615 unlike the array coil 50 according to the embodiment. For example, as illustrated in FIG. 7, in a case of forming a coil pattern in which coil elements 613 intersect with each other on the same plane, a solid wire 617 to be a jumper is required to be manually soldered after forming the coil pattern on the substrate so that adjacent coil elements 613 are prevented from being brought into electrical contact with each other. In the example illustrated in FIG. 7, to form a coil element 613b intersecting with coil elements 613a and 613c on the same plane, respective patterns constituting the coil elements 613b formed to be separated from each other on the substrate are required to be connected with each other by soldering a solid wire 617b. Similarly, to form a coil element 613d intersecting with the coil elements 613a and 613c on the same plane, respective patterns constituting the coil elements 613d formed to be separated from each other on the substrate are required to be connected with each other by soldering a solid wire 617d.

On the other hand, in the array coil 50 according to the present embodiment, as described above, the coil elements 513 and 533, which intersect with each other in a case of being formed on the same plane, are formed on the unit flexible substrates 511 and 531 different from each other. With this configuration, it is not required to perform work of manually soldering a solid wire to be a jumper to prevent the adjacent coil elements from being brought into electrical contact with each other for the coil elements formed on the unit flexible substrates.

That is, the array coil 50 according to the present embodiment can be formed by overlapping the unit flexible substrates 511 and 531 on which at least one of the coil elements 513 and 533 is formed. Due to this, the array coil 50 according to the present embodiment can be easily manufactured.

In the array coil 50 according to the present embodiment, unlike the coil pattern illustrated in FIG. 7, the coil elements 513 and 533 do not intersect with each other on the unit flexible substrates 511 and 531, that is, on the same plane. With this configuration, the adjacent coil elements 513 and 533 are not brought into electrical contact with each other unlike the array coil 60 in FIG. 7, so that it is not required to perform work of manually soldering the solid wire 617 to be a jumper after forming the coil pattern on the substrate. Accordingly, the array coil 50 according to the embodiment can be easily manufactured. Additionally, the adjacent coil elements are separated from each other, so that adjustment work for the array coil such as geometry decoupling can be easily performed, for example, and the array coil 50 can be manufactured more easily.

In the array coil 50 according to the present embodiment, the spacer 517 can be arranged at a position between the unit flexible substrates 511 and 531 where the coil elements 513 and 533, which intersect with each other in a case of being formed on the same plane, intersect with each other, that is, a position where the coil elements formed on the different unit flexible substrates 511 and 531 intersect with each other in plan view from the laminating direction. With this configuration, decoupling can be easily adjusted without influencing the arrangement of the coil elements 513 and 533.

The unit flexible substrates 511 and 531 can be overlapped to form the array coil 50, so that layout can be flexibly changed.

For example, the unit flexible substrates 511 and 531 to be overlapped may have the same arrangement of the coil elements 513 and 533. On the other hand, the unit flexible substrates 511 and 531 to be overlapped may have different arrangement of the coil elements 513 and 533.

For example, the thicknesses of the unit flexible substrates 511 and 531 to be overlapped may be caused to be the same, or caused to be different from each other.

For example, by laminating the unit flexible substrates 511 and 531, a plurality of the planar array coils 51 are formed. A unit substrate forming the planar array coil 51 may be the unit flexible substrates 511 and 531 having flexibility, or may be a unit substrate not having flexibility. The unit substrate forming the planar array coil 51 may be a combination of the unit flexible substrates 511 and 531 having flexibility and the unit substrate not having flexibility.

For example, the volume array coil 53 can be formed by winding the unit flexible substrates 511 and 531 having flexibility around the shaft 535.

By forming the coil elements 513 and 533, which intersect with each other in a case of being formed on the same plane, on different surfaces, it is not required to perform work of manually soldering the solid wire to be a jumper to prevent the adjacent coil elements from being brought into electrical contact with each other.

However, in a case of forming the coil elements on both surfaces of the substrate, the coil elements cannot be divided into two or more layers, so that all of the coil elements 513 and 533, which intersect with each other in a case of being formed on the same plane, cannot be formed on different surfaces in some cases. On the other hand, in the array coil 50 according to the present embodiment, three or more of the unit flexible substrates 511 and 531 can be laminated. Due to this, all of the coil elements 513 and 533, which intersect with each other in a case of being formed on the same plane, can be formed on the different unit flexible substrates 511 and 531 to implement a configuration in which all of the coil elements 513 and 533 do not intersect with each other on the same plane.

In a case of forming the coil elements on both surfaces of the substrate, positions of the coil element formed on one surface and the coil element formed on the other surface are fixed with respect to the thickness direction and a horizontal direction. On the other hand, in the array coil 50 according to the present embodiment, it is possible to laminate the unit flexible substrates 511 and 531 while adjusting them with respect to both of the thickness direction and the horizontal direction.

The array coil 50 according to the embodiment described above can be manufactured by forming a region in which the coil elements 513 and 533 are uniformly distributed by laminating the unit flexible substrates 511 and 531, and manually forming a region in which the coil elements 513 and 533 are nonuniformly distributed as described above with reference to the array coil 60 exemplified in FIG. 7.

A term of “processor” used in the above description means circuitry such as a CPU, a GPU, an ASIC, and a Programmable Logic Device (PLD), for example. The PLD includes a Simple Programmable Logic Device (SPLD), a Complex Programmable Logic Device (CPLD), and a Field Programmable Gate Array (FPGA). The processor implements a function by reading out and executing a computer program stored in storage circuitry. The storage circuitry in which the computer program is stored is a computer-readable non-transitory recording medium. Instead of storing the computer program in the storage circuitry, the computer program may be directly incorporated in circuitry of the processor. In this case, the processor implements a function by reading out and executing the computer program incorporated in the circuitry. Instead of executing the computer program, pieces of logic circuitry may be combined to implement a function corresponding to the computer program. Each processor in the present embodiment is not necessarily configured to be a single piece of circuitry, but may be configured to be one processor by combining a plurality of independent pieces of circuitry to implement the function. Furthermore, a plurality of constituent elements in FIG. 1 may be integrated into one processor to implement the function.

According to at least one embodiment described above, the array coil can be easily manufactured and adjusted.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

Regarding the embodiment described above, the following discloses Notes as an aspect and selective characteristics of the invention.

### (Note 1)

An array coil comprising:
a first substrate on which at least one coil element is formed; and
a second substrate different from the first substrate on which at least one coil element is formed, and is laminated on the first substrate, wherein
a first coil element formed on the first substrate intersects with a second coil element formed on the second substrate in plan view from a laminating direction from the second substrate laminated on the first substrate toward the first substrate.

### (Note 2)

A plurality of coil elements may be formed on at least one of the first substrate and the second substrate.

The coil elements do not intersect with each other on the substrate on which the coil elements are formed.

### (Note 3)

The array coil may further include a spacer arranged at a position between the first substrate and the second substrate where the first coil element intersects with the second coil element in the plan view.

### (Note 4)

The first substrate and the second substrate may have the same arrangement of the coil elements.

### (Note 5)

The first substrate and the second substrate may have different arrangement of the coil elements.

### (Note 6)

The first substrate and the second substrate may have different thicknesses.

### (Note 7)

The array coil may further include a plurality of substrates including the first substrate and the second substrate on each of which a plurality of coil elements are formed.

Each of the substrates may be further laminated on at least the other one of the substrates.

### (Note 8)

Each of the first substrate and the second substrate may be a flexible substrate having flexibility.

### (Note 9)

The array coil may be a planar array coil.

### (Note 10)

The array coil may be a volume array coil.

### (Note 11)

A manufacturing method for an array coil, the manufacturing method comprising:
forming at least one coil element on a first substrate;
forming at least one coil element on a second substrate different from the first substrate; and
laminating the second substrate on the first substrate so that a first coil element formed on the first substrate intersects with a second coil element formed on the second substrate in plan view from a laminating direction from the second substrate laminated on the first substrate toward the first substrate.

### (Note 12)

The manufacturing method may include a process of forming a plurality of coil elements on at least one of the first substrate and the second substrate.

The manufacturing method may also include a process of preventing the coil elements from intersecting with each other on the substrate on which the coil elements are formed.

### (Note 13)

The manufacturing method may further include a process of arranging an adjustment member at a position between the first substrate and the second substrate where the first coil element intersects with the second coil element in the plan view.

### (Note 14)

The manufacturing method may include a process of arranging the same coil elements on the first substrate and the second substrate.

### (Note 15)

The manufacturing method may include a process of arranging different coil elements on the first substrate and the second substrate.

### (Note 16)

The manufacturing method may include a process of preparing the first substrate and the second substrate with different thicknesses.

### (Note 17)

The manufacturing method may include a process of forming a plurality of coil elements on each of a plurality of substrates including the first substrate and the second substrate.

The manufacturing method may include a process of laminating each of the substrates on at least the other one of the substrates.

### (Note 18)

The manufacturing method may include a process of preparing each of the first substrate and the second substrate using a flexible substrate having flexibility.

### (Note 19)

The manufacturing method may include a process of preparing the array coil using a planar array coil.

### (Note 20)

The manufacturing method may include a process of preparing the array coil using a volume array coil.

What is claimed is:

1. An array coil comprising:
a first substrate on which at least one coil element is formed; and
a second substrate different from the first substrate on which at least one coil element is formed, and is laminated on the first substrate, wherein
a first coil element formed on the first substrate intersects with a second coil element formed on the second substrate in plan view from a laminating direction from the second substrate laminated on the first substrate toward the first substrate.

2. The array coil according to claim 1, wherein
a plurality of coil elements are formed on at least one of the first substrate and the second substrate, and
the coil elements do not intersect with each other on the substrate on which the coil elements are formed.

3. The array coil according to claim 1, further comprising:
a spacer arranged at a position between the first substrate and the second substrate where the first coil element intersects with the second coil element in the plan view.

4. The array coil according to claim 1, wherein the first substrate and the second substrate have the same arrangement of the coil elements.

5. The array coil according to claim 1, wherein the first substrate and the second substrate have different arrangement of the coil elements.

6. The array coil according to claim 1, wherein the first substrate and the second substrate have different thicknesses.

7. The array coil according to claim 1, comprising:
a plurality of substrates including the first substrate and the second substrate on each of which a plurality of coil elements are formed, wherein
each of the substrates is laminated on at least the other one of the substrates.

8. The array coil according to claim 1, wherein each of the first substrate and the second substrate is a flexible substrate having flexibility.

9. The array coil according to claim 1, wherein the array coil is a planar array coil.

10. The array coil according to claim 8, wherein the array coil is a volume array coil.

11. A manufacturing method for an array coil, the manufacturing method comprising:
forming at least one coil element on a first substrate;
forming at least one coil element on a second substrate different from the first substrate; and
laminating the second substrate on the first substrate so that a first coil element formed on the first substrate intersects with a second coil element formed on the second substrate in plan view from a laminating direction from the second substrate laminated on the first substrate toward the first substrate.
